(19) United States
(12) Patent Application Publication
(10) Pub. No.: US 2026/0294673 A1
(43) Pub. Date: Oct. 1, 2026

(54) PORTABLE SKIN COOLING DEVICE

(71) Applicant: STANG LIMITED, Hong Kong (CN)

(72) Inventor: Sidney TANG, Wanchai, Hong Kong (CN)

(21) Appl. No.: 19/122,494

(22) PCT Filed: Jun. 28, 2024
(86) PCT No.: PCT/CN2024/102457

§ 371 Date: Apr. 18, 2025

(30) Foreign Application Priority Data
Jul. 20, 2023 (CN) 202310895406.X

(51) Int. Cl. A61F 7/00; A61N 1/36; A61N 1/40; A61N 5/06

(52) U.S. Cl. CPC A61F 7/00; A61N 1/3603; A61N 1/40; A61N 5/0616; A61F 2007/0052; A61N 2005/0651

(57) ABSTRACT

The present invention belongs to the technical field of cold therapy devices and relates to a portable skin cooling device, comprising: a housing with an internal chamber; a thermoelectric module, fixed inside the inner housing's chamber, said thermoelectric module having a cooling surface; a cooling head, embedded on the housing's surface, said cooling head connected to the thermoelectric module's cooling surface; a power source, located inside the housing, said power source used to power the thermoelectric module; a PCB board, located inside the housing, said PCB board connected to both the thermoelectric module and the power source; wherein the thermoelectric module is used for cooling, the PCB board is provided with a central control module, said central control module used to control the cooling temperature of the thermoelectric module, and the cooling head is used to contact the skin for cold therapy.

### TECHNICAL FIELD

The present invention belongs to the technical field of cold therapy devices and relates to a portable skin cooling device.

### BACKGROUND OF THE INVENTION

Traditional methods of treating skin conditions and relieving itching involve using medications, steroids, hand creams, and lotions. These methods are slow to respond, troublesome, and can cause various side effects. Relieving itching through cold regulation is a non-pharmaceutical natural technique. When the skin is exposed to harmless cold stimulation, it stimulates temperature-sensitive cells, thereby reducing itching. Research indicates that heat increases itching, while cold can alleviate it. Cold stimulation needs to be intense to effectively suppress irritation, inflammation, pain, and itching. Cold temperature can numb nerves and reduce blood flow to the area, thereby reducing itching and pain. Ice can also reduce swelling and discomfort associated with various skin rashes or allergic symptoms.

However, current applications generally use ice or ice packs for cold therapies. Ice or ice packs need to be prepared in advance and stored in containers, making them inconvenient for immediate use. Ice used directly on the skin has too low temperature, risking cold burn, and ice or ice packs exposed to the atmospheric environment are prone to condensation and melting, making them extremely inconvenient to use.

### CONTENT OF THE INVENTION

The purpose of the present invention is to address the shortcomings of existing technologies by providing a portable skin cooling device that uses thermoelectric module for cold therapy, used to relieve itching, reduce pain and inflammation, minimize the risk of low-temperature cold burn, and be convenient to use and carry.

To achieve the above objectives, the present invention adopts the following technical solution:
A portable skin cooling device, comprising:
A housing, with a chamber inside the housing;
A thermoelectric module, fixed inside the housing's chamber, said thermoelectric module having a cooling surface;
A cooling head/tip/end, embedded on the surface of the housing, said cooling head connected to the cooling surface of the thermoelectric module;
A power source, located inside the housing, said power source used to supply power to the system;
A PCB board, located inside the housing, said PCB board connected to both the thermoelectric module and the power source;
Wherein, the thermoelectric module is used for cooling, the PCB board is provided with a central control module, said central control module is used to control the cooling temperature of the thermoelectric module, and the cooling head is used to contact the skin for cold therapy.

Further, the thermoelectric module includes a thermoelectric cooler.

Further, the thermoelectric module is composed of one or multiple thermoelectric coolers arranged in cold and or hot surfaces stacked or arranged in a planar array.

Further, it includes a cooling temperature sensor, said cooling temperature sensor located inside the housing and connected to the PCB board, used to monitor the temperature data of the cooling head and feedback to the central control module.

Further, a heat sink module is provided inside the housing, the thermoelectric element includes a cooling surface and a heating surface, said heat sink module connected to the heating surface.

Further, the heat sink module includes a heat pipe, connected to the heating surface of the thermoelectric module inside the housing, with phase change material sealed inside the heat absorption tube.

Further, it includes a heat sink module temperature sensor, said heat sink module temperature sensor fixed inside the housing and connected to the central control module, used to monitor the temperature of the heat sink module and feedback to the central control module.

Further, it includes a vibration module, said vibration module fixed inside the housing and connected to the central control module.

Further, it includes light therapy LED and radio frequency electrodes.

Further, it includes electrotherapy electrodes, said electrotherapy electrodes fixed on the surface of the housing and connected to the central control module, used for electrotherapy.

Referring to the technical solution of this invention, advantages over existing technologies include
1. Excellent skin cooling effect, through the cooling surface of the thermoelectric module and cooling head, able to quickly lower skin temperature, reduce discomfort, itch and pain, and help reduce swelling and soothe the skin.
2. Precise temperature control, with the aid of the central control module and cooling temperature sensor, it can precisely control the cooling temperature, avoiding situations of over-cooling or insufficient cooling, providing safe and effective cooling.
3. Effective heat dissipation, through the heat sink module and phase change material, able to effectively absorb and release heat, maintain stable working temperature of the device and thermoelectric module, improve service life and safety, with a fanless design, no operational noise, no moving parts and good waterproof and dust-proof performance.
4. Multi-functional application, the device provides options for light therapy and electrotherapy, can meet different skin problem needs, offering more comprehensive treatment effects.
5. Vibration module increases comfort, the vibration module inside the device can provide massage and soothing effects, increasing comfort during use, making the entire treatment process more pleasant.

Other features and advantages of the invention will be elaborated in the subsequent specification and will become partly apparent from the specification or understood through practicing the invention. The objectives and other advantages of the invention can be achieved and obtained through the specific structures pointed out in the written specification and accompanying drawings.

### BRIEF DESCRIPTION OF THE DRAWINGS

The following detailed description of the invention is made in conjunction with the accompanying drawings to make the above advantages of the invention more clear.

FIG. 1 is an exploded view of a portable skin cooling device according to the invention;

FIG. 2 is a connection block diagram of a portable skin cooling device according to the invention;

FIG. 3 is a temperature control schematic diagram of a portable skin cooling device according to the invention.

### DETAILED DESCRIPTION OF EMBODIMENTS

The following details the embodiments of the invention, with examples shown in the accompanying drawings, where identical or similar numbers throughout represent identical or similar elements or elements with identical or similar functions. The embodiments described with reference to the drawings are exemplary and intended to explain the invention, not to be understood as limiting the invention.

In the description of the invention, it should be understood that terms such as “length”, “width”, “up”, “down”, “front”, “back”, “left”, “right”, “vertical”, “horizontal”, “top”, “bottom”, “inside”, “outside” indicate orientation or positional relationships based on the drawings, and are merely for convenient description and simplification, not indicating or implying that the described device or element must have a specific orientation, be constructed and operated in a specific orientation, and therefore should not be understood as limiting the invention.

Moreover, terms “first” and “second” are used only for descriptive purposes and should not be understood as indicating or implying relative importance or implicitly specifying the number of technical features. Thus, features defined with “first” and “second” may explicitly or implicitly include one or more such features. In the description of the invention, “multiple” means two or more, unless specifically defined otherwise.

In the embodiments of the invention, unless otherwise specifically stipulated and limited, terms such as “install”, “connect”, “couple”, “fix” should be understood broadly. For example, it can be a fixed connection, or a detachable connection, or integrated; it can be a mechanical connection or an electrical connection; it can be a direct connection or an indirect connection through an intermediate medium, or can be an internal communication between two elements or an interaction relationship between two elements. For those skilled in the art, the specific meaning of these terms in the invention can be understood according to specific circumstances.

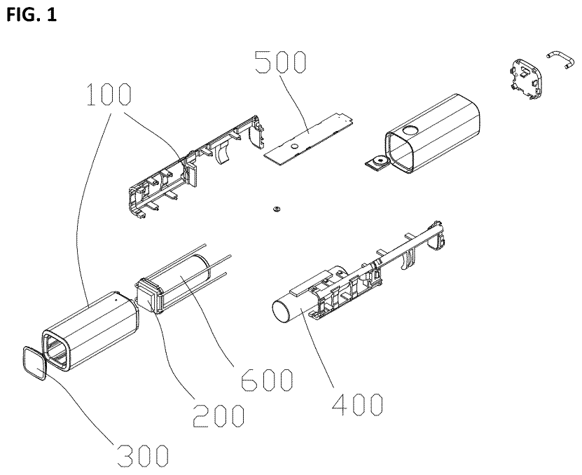

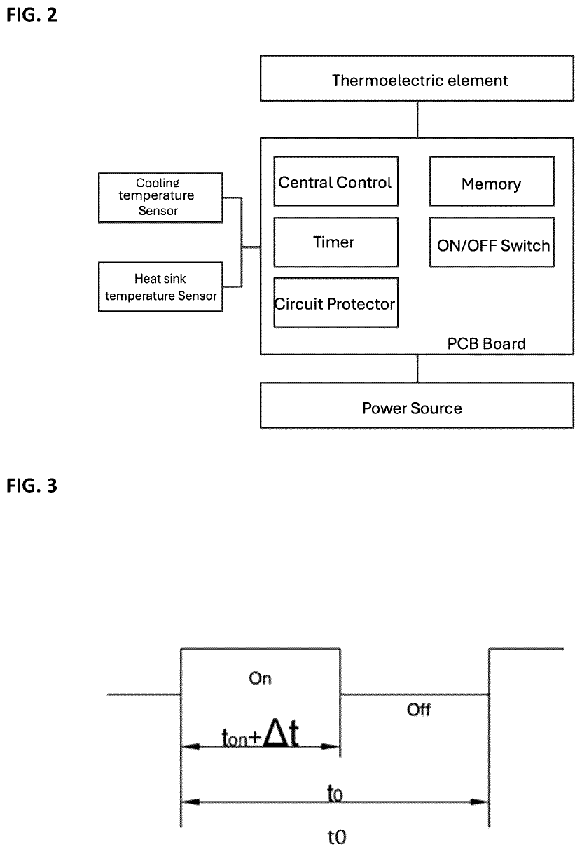

Referring to FIGS. 1-3, a portable skin cooling device comprises:
Housing 100, with a chamber inside the housing;
Thermoelectric module 200, fixed inside the housing's chamber, said thermoelectric module 200 having a cooling surface;
Cooling head 300, embedded on the surface of the housing 100, said cooling head 300 connected to the cooling surface of the thermoelectric module 200;
Power source 400, located inside the housing 100, said power source 400 used to supply power to the thermoelectric module 200;
PCB board 500, located inside the housing 100, said PCB board 500 connected to both the thermoelectric module 200 and the power source 400;
Wherein, the thermoelectric module 200 is used for cooling, the PCB board 500 is provided with a central control module, said central control module is used to control the cooling temperature of the thermoelectric module 200, and the cooling head 300 is used to contact the skin for cold therapy.

The power source 400 is a DC power source 400. When cold therapy is needed, the power source 400 supplies power to the thermoelectric module 200, and during charging, the power protection circuit on the PCB protects the power source. The thermoelectric module 200 performs cooling, bringing the cooling head 300 close to the skin, provides continuous cooling to maintain low temperature, which can compensate for the temperature rebound from skin surface. The cooling effect stimulates the skin and constricts blood vessels and pores, thereby reducing blood flow and providing immediate and temporary relief and comfort to reduce irritation, pain, or itching.

The cooling head 300 uses non-allergenic and high thermal conductivity materials, such as stainless steel, copper or aluminum. Since the cooling head 300 directly contacts the skin for cold therapy, non-allergenic materials are used to avoid allergic reactions for special skin types. At the same time, to optimize the user experience and make the cold therapy effect more uniform, a highly conductive material is used. This avoids problems such as uneven cold therapy or poor temperature transfer efficiency.

During cold therapy, frostbite prevention must also be considered. There are two ways to prevent frostbite: First, constant power mode, where the thermoelectric element 200 is DC-driven, and the working current is controlled by the central control module to achieve constant power, making the cooling amount slightly larger than the human body's heat rebound, avoiding low-temperature frostbite. Second, timed mode, where the timing unit on the PCB sets a continuous cold therapy time threshold, and when the continuous operation time reaches the threshold, the thermoelectric module 200 automatically cuts off power to prevent situations such as the user falling asleep and causing continuous cold therapy that might affect health.

Additionally, the storage unit on the PCB can be used to preprogram different modes according to different usage requirements, setting the optimal cold therapy time and thermoelectric module 200. Different cooling power for needs such as anti-itch, pain relief, and swelling reduction. This allows users to set and switch modes (single button).

In this embodiment, for ease of holding during use, the housing 100 is tubular, with the cooling head 300 embedded at the housing's end. The cooling head 300 can also adopt different shapes, geometries, circular, concave or convex, or roller forms. The cooling head 300 can have different sizes, suitable for various inflammations, reducing pain and itching, and promoting collagen production.

In this embodiment, the thermoelectric module 200 includes a thermoelectric cooler. A thermoelectric cooler is a solid-state heat pump that creates a temperature difference by transferring heat between two electrical junctions when an electric current is passed through thus cooling objects. It uses bismuth telluride compound semiconductor elements, generating voltage when two different metals are connected, and then using current to transfer heat from one side of the device to the other. This process is called the Peltier effect, which can precisely control temperature and reduce energy consumption compared to traditional methods. Compared to other cooling devices, thermoelectric coolers have the following advantages:
1. No refrigerant required: Thermoelectric cooling devices do not need traditional refrigerants, making it an environmentally friendly and sustainable cooling technology.
2. High reliability: Thermoelectric cooling devices are composed of semiconductor materials with no moving parts, thus offering high reliability. Thermoelectric cooler is not easily damaged or prone to failure.
3. Compact size: Thermoelectric cooling devices are relatively small and compact, suitable for small or portable devices.
4. Flexible control: Thermoelectric cooling devices feature quick response and precise temperature control. By adjusting voltage or current, precise cooling effects can be achieved to meet different needs and treatment requirements.
5. Low energy consumption: Compared to traditional cooling devices, thermoelectric cooling devices consume less energy. They only consume energy when cooling is needed and do not require additional energy for refrigerant circulation, thus saving energy.
6. Low noise: Thermoelectric cooling devices have no moving parts, resulting in low noise levels during operation, providing a quieter and more comfortable cold therapy experience.

In this embodiment, the thermoelectric module 200 is composed of multiple thermoelectric coolers arranged in stacked hot and cold surfaces or in a planar array.

Thermoelectric module stacking: By stacking multiple thermoelectric coolers together, the thermoelectric power and capacity can be increased. The principle of thermoelectric cooler stacking involves connecting multiple thermoelectric coolers in a specific manner to allow heat transfer between thermoelectric modules. Each thermoelectric module has two contact surfaces, a cold surface and a hot surface. By stacking multiple thermoelectric coolers, a structure with alternating hot and cold surfaces can be formed. When current passes through each TEC module, a heat difference is generated at each contact surface. This enables a larger temperature difference and higher cooling or heating capability. Thermoelectric cooler stacking can also provide higher thermoelectric power, efficiency and a more precise temperature control range.

Planar array arrangement: This refers to arranging multiple Thermoelectric cooling modules on a single plane, forming a two-dimensional array structure. Each thermoelectric cooling module typically has only one contact surface, either cold or hot. Planar array arrangement is used for scenarios requiring a larger cooling area.

In this embodiment, it also includes a cooling temperature sensor, which is located inside the housing 100 and connected to the PCB board 500. The cooling temperature sensor is used to monitor the temperature data of the cooling head 300 and feed it back to the central control module. Since the cooling head 300 is a component that directly contacts the skin, there is heat conduction between the cooling head 300 and the Thermoelectric module 200, which introduces some delay in temperature control, affecting the accuracy and response speed of temperature control. To improve the safety and temperature control precision of the cold therapy, the cooling temperature sensor directly monitors the temperature of the cooling head 300. The central control module can adjust the power of the electric heating element 200 in real-time based on the feedback from the cooling temperature sensor, achieving constant temperature control and avoiding sudden temperature changes, ensuring a comfortable user experience.

Through the feedback from the temperature sensor, the central control module can control the cooling temperature based on variable pulse control. When the device is started, to quickly reach the preset cold temperature of the cooling head 300, the pulse control extends the power on duration to accelerate temperature reduction. It also adjusts the control pulse real-time through the feedback from the cooling temperature sensor. Within the pulse cycle to, the initial cooling time is t on, and the adjustment time is At. If the real-time temperature is higher than the threshold temperature, At is a positive number; if the real-time temperature is lower than the threshold temperature, At is a negative number.

In this embodiment, a digital display can be provided on the surface of the housing 100. The digital display is connected to the central control module and can be used to display the real-time temperature of the cooling head 300, modes and the remaining power of the power source 400, facilitating the user's understanding of the cooling head's cooling situation.

In this embodiment, a heat sink module is set inside the housing 100. The thermoelectric module 200 includes a cooling surface and a heating surface, and the heat sink module is connected to the heating surface. A thermoelectric cooler is a device that transfers heat energy from one side to another to cool or heat objects. During the instruction process, the opposite side of the thermoelectric cooling surface is the heating surface. Its cooling principle involves electric current passing through P-type and N-type semiconductor materials connected on both sides, causing one side to absorb heat while the other side releases heat, thereby achieving a cooling effect. In the thermoelectric cooling process, the heat sink on the hot side impacts the cooling effect. This is because the TEC's cooling effect is achieved by transferring heat from one side to another. If the other side cannot effectively dissipate heat, heat will accumulate inside the thermoelectric module, reducing the temperature difference and weakening the cooling effect.

Therefore, to ensure the thermoelectric cooling effect, it is necessary to ensure effective heat dissipation on the hot side.

This can be achieved by using heat sinks, fans, or other cooling devices to increase the heat dissipation surface area and improve heat dissipation efficiency. Additionally, optimizing the selection of heat dissipation materials and surface treatment can be considered to enhance the heat dissipation effect.

In this embodiment, the heat sink module includes a heat absorption tube 600, connected to the heating surface of the electric power source 400 fixed inside the housing 100, with a phase change material sealed inside the heat absorption tube 600. Conventional heat dissipation solutions are plentiful, such as using heat sinks, fans, or other cooling devices to increase heat dissipation surface area and improve heat dissipation efficiency.

In this embodiment, the heat pipes are heat dissipation elements that utilize the phenomenon of liquid absorbing heat when transforming into gas and releasing heat when transforming back to gas, enabling rapid heat transfer from one location to another.

Compared to general metal sheets, heat pipes have the following advantages:
1. Fast heat transfer speed, effectively removing heat.
2. Long heat transfer distance, effectively dispersing heat.
3. Able to transfer heat without any moving parts, making heat pipes quiet, noiseless, maintenance-free, and highly reliable.

The advantages of thermoelectric cooling devices include low noise, whereas conventional cooling methods often generate significant noise and require additional housing openings, compromising the housing's dust and water resistance. The phase change material is encapsulated in a brass tube to increase exposure to the surrounding environment, improving heat transfer efficiency. The heat sink's temperature rises with the thermoelectric module operating temperature until a certain point, at which the phase change material begins to melt and absorb the system's excess heat, enabling more effective heat absorption. Once all phase change material has melted and reached the desired temperature, it needs to rest and prevent overheating. It begins to re-solidify, returning to its original state, with reusable heat absorption and dissipation capabilities.

Using phase change material for heat absorption in the absorption tube offers the following advantages compared to traditional air cooling:
1. No fan or mechanical parts are required, thus generating no noise.
2. Phase change materials have high heat capacity and thermal conductivity, enabling rapid heat absorption and release. Compared to fan cooling, phase change material cooling can more effectively lower device temperature and improve heat dissipation.
3. Phase change material cooling requires no additional power supply, producing no extra energy consumption. Compared to fan cooling, it is more energy-efficient and improves the usability of portable devices.
4. Phase change material cooling has no mechanical components, thus lower maintenance costs. Unlike fan cooling, it requires no periodic cleaning or fan replacement, reducing device maintenance work and expenses.

Phase change materials can be hydrated salts, hydrated salt mixtures, paraffin compounds, organic compounds such as decanoic acid, lauric acid, stearic acid, and other fatty acids. The set melting point of the phase change material will be determined by usage conditions, operating temperature, and ambient temperature.

In this embodiment, a heat sink module temperature sensor is also included, fixed inside the housing 100 and connected to the central control module. The heat sink module temperature sensor is used to monitor the temperature of the heat sink module and feed back to the central control module. In thermoelectric cooling applications, monitoring the heat sink temperature is crucial, as it directly affects thermoelectric cooler heat dissipation efficiency. When the heat sink temperature is too high, TEC cooling capacity decreases, leading to reduced cooling effects. By monitoring heat sink temperature, the heat dissipation system can be timely adjusted and optimized to ensure efficient thermoelectric cooling. Additionally, excessively high heat sink temperatures may cause overheating of the heat sink and surrounding components, potentially leading to failures or damage. Monitoring heat sink temperature allows timely detection of temperature anomalies, enabling appropriate measures to protect system safety. The lifespan and stability of thermoelectric cooler are also closely related to heat sink temperature. Excessively high heat sink temperatures cause thermoelectric cooler operating temperature to rise, accelerating aging and reducing lifespan. Temperature fluctuations can also affect thermoelectric cooler stability and performance. By monitoring heat sink temperature, thermoelectric cooler working temperature can be controlled within an appropriate range, extending its lifespan and improving stability.

In another embodiment, a vibration module is also included, fixed inside the housing 100 and connected to the central control module. The vibration module is installed in the housing 100, producing vibration of the housing 100 and cooling head 300 to achieve a massage effect on the skin Cold therapy can effectively reduce pain and inflammation, while the vibration from vibration module can alleviate itch and pain perception by stimulating the skin and muscles, thereby diverting attention. The combined use of both can provide better pain relief.

The vibration module's vibration can promote local blood circulation and increase blood flow. Meanwhile, cooling can constrict blood vessels and reduce local blood flow. By combined use, better blood circulation can be achieved during cold therapy.

Combining cold therapy and vibration can accelerate injury site recovery. Cold therapy can reduce inflammation and swelling, alleviating tissue damage. The vibration module can promote local blood circulation, enhance muscle and tissue nutrient supply, and facilitate recovery and repair processes.

The vibration module's vibration can help relax tense muscles, relieving muscle fatigue and tension. Cold therapy can alleviate muscle pain and discomfort by lowering local temperature. Combined use can provide better muscle relaxation effects.

In another embodiment, LED is also included, used for light therapy. The LEDs can be a red light LED, a blue light LED, and any coloured combinations thereof.

Cold therapy can reduce inflammation and swelling, and light therapy is also proven to have anti-inflammatory effects. Combining cold therapy and light therapy can more effectively alleviate inflammatory responses, reducing pain and discomfort. Light therapy is widely applied in tissue repair and trauma rehabilitation. It can promote cell metabolism, increase ATP cell energy production, and accelerate wound healing and tissue repair processes. Cold therapy can reduce blood flow to the local area, reducing inflammatory responses and providing a better environment for tissue repair.

Light therapy has been proven to have antibacterial effects, effectively killing bacteria and reducing infection risks. Combining cold therapy and light therapy can provide additional antibacterial effects during cold therapy, beneficial for wound healing after trauma or surgery.

In another embodiment, radiofrequency electrodes are also included, fixed on the surface of the housing 100 and connected to the central control module. Radiofrequency electrodes generate heat to stimulate deep skin tissue, promoting collagen regeneration and tissue tightening. However, radiofrequency treatment may cause certain skin surface heat sensation and discomfort.

In this case, cold therapy can help cool the skin, reduce discomfort, and provide additional soothing effects. Cold therapy can alleviate heat sensation by lowering skin temperature, reducing redness and local inflammation.

When using radiofrequency electrodes and cold therapy together, the general steps are:
1. Perform radiofrequency treatment: Place radiofrequency electrodes correctly on the skin and choose appropriate energy and time as needed.
2. Cold therapy: After radiofrequency treatment, gently press or slide the thermoelectric cooling surface on the treatment area to cool the skin.
3. Using radiofrequency electrodes in combination with cold therapy can help alleviate the heat sensation and discomfort during radiofrequency treatment, while simultaneously providing a soothing effect.

In another embodiment, electrotherapy electrodes are also included, fixed on the surface of the housing 100 and connected to the central control module, used for electrotherapy.

Electrode electrotherapy is a method of pain relief through electrical stimulation of nerve conduction. Cold therapy can reduce local temperature to reduce inflammation and swelling, while electrode electrotherapy can stimulate nerves, interfere with pain signal transmission, and thus reduce pain sensation.

Electrotherapy can stimulate muscle fibers, reducing muscle tension and spasms. Cold therapy can reduce muscle pain and discomfort by lowering local temperature. Combined use can provide better muscle relaxation effects.

Electrotherapy can promote local blood circulation and increase blood flow through electrical stimulation. Cold therapy can constrict blood vessels and reduce local blood flow. Combined use can provide better blood circulation promotion during cold therapy.

Finally, it should be noted that the above descriptions are merely preferred embodiments of the present invention and should not be used to limit the invention. Although the present invention has been described in detail with reference to the foregoing embodiments, those skilled in the art can still modify the technical solutions described in the foregoing embodiments or make equivalent replacements of some technical features. Any modifications, equivalent substitutions, or improvements made within the spirit and principles of the present invention should be included within the scope of protection of the invention.

1. A portable skin cooling device comprising:
housing (100), wherein the housing (100) has a chamber formed inside;
thermoelectric module (200), fixed within the chamber of the housing (100), wherein the thermoelectric module (200) is provided with a cooling surface;
cooling head (300), embedded on the surface of the housing (100), wherein the cooling head (300) is connected to the cooling surface of the thermoelectric module (200);
power source (400), located inside the housing (100), wherein the power source (400) is used to supply power to the thermoelectric module (200);
PCB board (500), located inside the housing (100), said PCB board (500) connected to both the thermoelectric module (200) and the power source (400);
wherein, the thermoelectric module (200) is used for cooling, the PCB board (500) is provided with a central control module, the said central control module is configured to control the cooling temperature of the thermoelectric module (200), and the cooling head (300) is used for contact with the skin for cold therapy.

2. The portable skin cooling device according to claim 1, characterized in that the thermoelectric module (200) comprises a thermoelectric cooler.

3. The portable skin cooling device according to claim 2, characterized in that the thermoelectric module (200) comprises one or multiple thermoelectric coolers arranged by stacking cold and hot surfaces or in a planar array.

4. The portable skin cooling device according to claim 1, further comprising a cooling temperature sensor, the cooling temperature sensor being located inside the housing (100) and connected to the PCB board (500), wherein the cooling temperature sensor is used to monitor temperature data of the cooling head (300) and feed back to the central control module.

5. The portable skin cooling device according to claim 1, characterized in that the housing (100) is provided with a heat sink module, the thermoelectric module (200) comprises a cooling surface and a heating surface, and the heat sink module is connected to said heating surface.

6. The portable skin cooling device according to claim 5, characterized in that the heat sink module comprises a heat pipe (600), connected to the heating surface of the thermoelectric power source (400) fixed inside the housing (100), with a phase change material sealed inside the heat pipe (600).

7. The portable skin cooling device according to claim 5, further comprising a heat sink module temperature sensor, said heat sink module temperature sensor fixed inside the housing (100) and connected to the central control module, said heat sink module temperature sensor used to monitor the temperature of the heat sink module and feed back to the central control module.

8. The portable skin cooling device according to claim 1, further comprising a vibration module, said vibration module fixed inside the housing (100) and connected to the central control module.

9. The portable skin cooling device according to claim 1, further comprising LED light therapy module and radio frequency therapy electrodes.

10. The portable skin cooling device according to claim 1, further comprising electrotherapy electrodes, said electrotherapy electrodes fixed on the surface of the housing (100) and connected to the central control module, said electrotherapy electrodes used for electrotherapy.
